(19) United States
(12) Patent Application Publication
(10) Pub. No.: US 2014/0316404 A1
(43) Pub. Date: Oct. 23, 2014

(54) Electrosurgical Device

(71) Applicant: SÖRING GMBH, Quickborn (DE)

(72) Inventors: Florian Neumann, Quickborn (DE); Klaus Zobawa, Quickborn (DE)

(21) Appl. No.: 14/354,726

(22) PCT Filed: Oct. 31, 2012
(86) PCT No.: PCT/EP2012/071592

§ 371 Date: Apr. 28, 2014

(30) Foreign Application Priority Data
Oct. 31, 2011 (DE) 10 2011 085 501.7

(51) Int. Cl. A61B 18/12

(52) U.S. Cl. CPC A61B 18/1233; A61B 18/1477
USPC 606/34

(57) ABSTRACT

An electrosurgical device comprises a first signal generator and a second signal generator, wherein the signal generators are configured to generate an electrical signal that can be transmitted to a patient via electrodes. A control module is provided in which an output value of the first signal generator and an output value of the second signal generator are combined to form an aggregate output value, and which determines whether the aggregate output value exceeds a predetermined threshold value. A method for operating such an electrosurgical device ensures that the patient is not inadvertently treated with an excessively high electrical output.

### BACKGROUND

The invention relates to an electrosurgical device comprising a first signal generator and a second signal generator. The signal generators are designed to generate an electrical signal that can be transmitted to a patient via electrodes. The invention additionally relates to a method for operating such an electrosurgical device.

In electrosurgery, the tissue of a patient is treated by electrical energy that is transmitted to the tissue via electrodes. The signal generator serves to generate a suitable electrical signal. The effect on the tissue depends on what quantity of energy is transmitted to the tissue. By way of example, a flow of blood can be staunched or tissue can be severed by means of electrosurgery.

It is often the case that during operations a plurality of electrosurgical instruments are used simultaneously. In this regard, for instance, one electrosurgical instrument can be used to sever tissue at one site, while other electrosurgical instruments simultaneously staunch the flow of blood at other sites. When a plurality of electrosurgical instruments are used, care must be taken to ensure that the total amount of electrical energy introduced into the body by the plurality of electrosurgical instruments does not become too high. In this regard, on average over 1 s not more than 400 W should be introduced into the body.

Hitherto it has been necessary for the operation personnel to set the signal generators manually such that the permissible limits are complied with. In other words, it is necessary for one person to have an overview of how many electrosurgical instruments are active and with what power said instruments are operated. In the case of operations involving the cooperation of a plurality of persons, it is not very easy to maintain said overview. There is the risk of too many electrosurgical instruments being operated in parallel with one another, and of the patient being treated with excessively high power.

An electrosurgical device and an associated method are presented for which the risk of the patient being treated with excessively high power is reduced.

### SUMMARY

Briefly stated, the electrosurgical device comprises a control module, in which a power value of the first signal generator and a power value of the second signal generator are combined to form a total power value. The control module ascertains whether the total power value exceeds a predefined threshold value.

By virtue of the automatic combination of the power values in the control module, it becomes easier for the operation personnel to comply with the prescribed limits. If the control module ascertains that the predefined threshold value has been exceeded, it is possible for the activation of further electrosurgical instruments to be dispensed with or for the power to be reduced in a suitable manner.

The electrosurgical device can comprise just two signal generators. However, the advantages are manifested to an increased extent if more than two signal generators are provided. The more signal generators there are, the more difficult it becomes for the operation personnel to maintain the overview. By way of example, a piece of tissue can be separated by means of two signal generators, while at the same time the flow of blood is staunched at different sites by means of three further signal generators. In this case, the control module can combine the power values of all the signal generators to form a total power value.

The control module can be designed such that a signal for the operation personnel is output after the predefined threshold value has been exceeded. The signal can be optical or acoustic, for example. The operation personnel can then react in a suitable manner and deactivate one or more signal generators or reduce the power.

As an alternative or in addition thereto, the control module can be designed such that, after the predefined threshold value has been exceeded, said control module outputs a control command directed to one or more signal generators. The signal generator can be deactivated by the control command. Alternatively, a power limit can be set for the signal generator. In this way, the control module can automatically ensure that the electrical power output to the patient remains below predefined limits.

The signal generators can be designed such that they can be set to different power levels, wherein each power level defines a different maximum power for the signal generator. This does not mean that the signal generator is always operated with the maximum possible power in the power level, rather the power output of the signal generator is variable within the power range defined by the maximum power.

The power values processed in the control module can relate to the power level of the signal generators. If the sum of the maximum possible powers already lies below the threshold value, then the sum of the actual power instantaneously present is less than the threshold value to an even greater extent. The threshold value is exceeded in this embodiment if the sum of the power levels, that is to say the sum of the maximum possible powers within the power levels, lies above the threshold value.

If the power values relate solely to the power level of the signal generator, then the actual power instantaneously present will lie far below the threshold value the vast majority of the time, since it is unlikely that all the signal generators will be operated with the maximum power of the relevant power level at the same time. The electrosurgical device can therefore be designed such that the power values relate to the actual power of the signal generators that is instantaneously output.

The electrosurgical device can be designed such that the instantaneous power is monitored permanently. Alternatively, a two-stage procedure can be provided, in which firstly the power levels are monitored. It is only if the power levels in total have exceeded the threshold value that a transition is made to monitoring the instantaneous powers. This has the advantage that at the first stage each signal generator can definitely supply the power requested from it within the power level. At the second stage, it is necessary to reckon with the fact that one or more signal generators are temporarily subject to a power restriction.

If the fact that the threshold value has been exceeded is ascertained, it is necessary to decide in what way the electrosurgical device can be operated such that the power output to the patient remains within the envisaged limits. If the exceedance was caused for example by the fact that a further signal generator was supposed to be put into operation, this signal generator can be prevented from commencing operation. The previous signal generators then remain in operation, the power values of said previous signal generators in total lying below the threshold value. Alternatively, the relevant signal generator can indeed be allowed to commence operation, but a power limit lying below the maximum power of the power level can be set for said signal generator.

On each occasion when the threshold value is exceeded, the question arises as to for which of the signal generators a power limit is set or which signal generator is deactivated. An order can be defined by a priority being assigned to the signal generators in the form of a master-slave configuration. The higher-priority signal generators can then be operated with the full desired power, while a power limit is set for the lower-priority signal generators, or the latter are deactivated.

In addition or as an alternative thereto, provision can also be made for mutual monitoring of the signal generators to take place. For this purpose, a plurality of control modules can be provided, wherein a control module is preferably assigned to each signal generator. The total power value is determined in each control module. This affords the possibility of ascertaining in each signal generator, before a change in the power, whether the power change is still possible without exceeding the threshold value. If this is not the case, the power change is not performed. It is thereby automatically evident for which of the signal generators the power is restricted.

A method is provided for operating an electrosurgical device comprising a first signal generator and a second signal generator. The method involves providing a power value of the first signal generator and a power value of the second signal generator. The power values are combined to form a total power value. The method involves ascertaining whether the total power value exceeds a predefined threshold value.

The method can be developed with further features described above with reference to the electrosurgical device.

### BRIEF DESCRIPTION OF THE DRAWINGS

The invention is described by way of example below on the basis of advantageous embodiments with reference to the accompanying drawings, in which:

FIG. 1 shows a first embodiment of an electrosurgical device;

FIG. 2 shows a second embodiment of an electrosurgical device;

FIG. 3 shows a third embodiment of an electrosurgical device;

FIG. 4 shows an illustration corresponding to FIG. 2, wherein the device is designed for a bipolar application.

### DETAILED DESCRIPTION

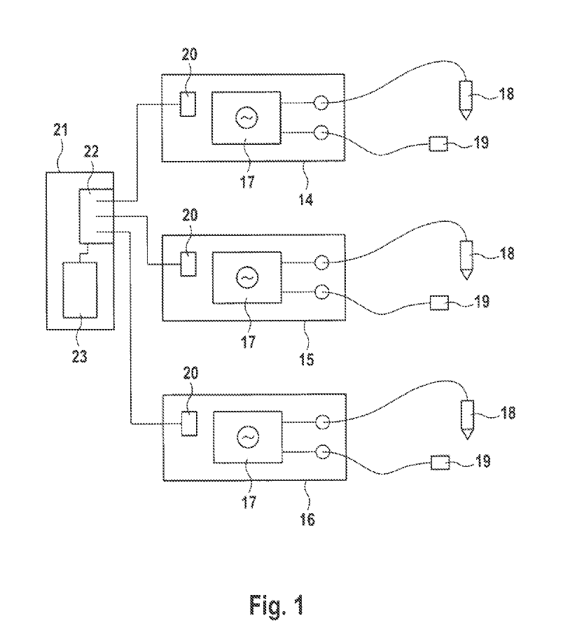

An electrosurgical device in FIG. 1 comprises a first electrosurgical instrument 14, a second electrosurgical instrument 15 and a third electrosurgical instrument 16. Each electrosurgical instrument 14, 15, 16 is equipped with a signal generator 17 designed to generate a radio-frequency electrical signal having a frequency of between 500 kHz and 3000 kHz, for example. An active electrode 18 and a neutral electrode 19 are in each case connected to the signal generators.

The neutral electrode 19 is connected to the patient's body. The active electrode 18 forms the manual instrument that the surgeon works with. If the active electrode 18 is brought into contact with the patient's tissue, an electric current flows through the patient's body to the neutral electrode 19. In direct proximity to the active electrode 18, the electric current has a considerable effect on the tissue, this effect rapidly being lost with increasing distance from the active electrode 18. This locally delimited effect of the active electrode 18 is used for example to sever the tissue or to staunch a flow of blood. Since the surgeon only guides the active electrode by means of the manual instrument, this application is designated as monopolar.

The plurality of electrosurgical instruments 14, 15, 16 make it possible to treat the patient simultaneously with a plurality of active electrodes 18, wherein the electrical signal generated by the signal generator 17 can be set and adapted for the individual active electrodes 18 independently of one another. In this regard, by way of example, it is possible to use the electrosurgical instrument 14 with high power to sever tissue. At the same time, it is possible to operate the electrosurgical instruments 15, 16 with lower power in order to coagulate blood. In this case, it must be ensured that the three electrosurgical instruments 14, 15, 16 as considered together on average over 1 s do not output more than a power of 400 W to the patient. It would therefore be permissible, for example, to operate the electrosurgical instrument 14 with a power of 250 W and at the same time the electrosurgical instruments 15, 16 with a power of 75 W, such that the total obtained is precisely a power of 400 W. By contrast, simultaneously operating all the electrosurgical instruments 14, 15, 16 with full power is impermissible.

The electrosurgical instruments 14, 15, 16 can respectively be set to different power levels of 75 W, 150 W and 250 W, for example, wherein the power indication relates to the maximum power of the power level. Each electrosurgical instrument 14, 15, 16 comprises an indicator 20, which communicates the power level currently set to a control module 21 via a line. In a logic component 22 of the control module 21, the three power values communicated by the electrosurgical instruments 14, 15, 16 are added up to form a total power value and compared with a threshold value of 400 W stored in the logic component 22. If the total power value exceeds the threshold value, a signal is passed to a command transmitter 23. The command transmitter 23 communicates a control command to the electrosurgical instrument from which the last change in the power level originates. The electrosurgical instrument infers from the control command that the last change made in the power level is not possible. The operation of the electrosurgical instrument is continued with the power level previously set.

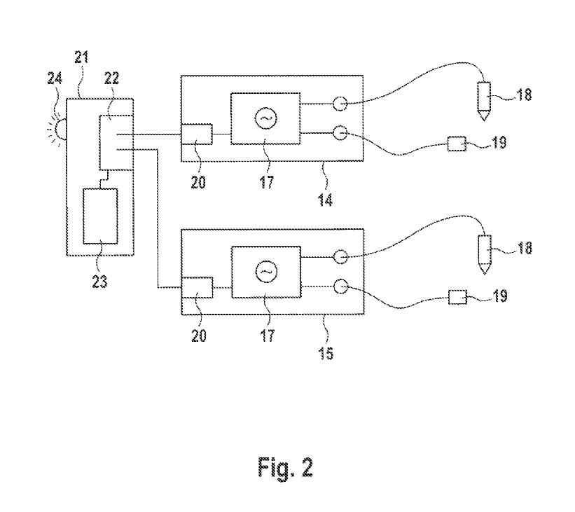

In the embodiment in accordance with FIG. 2, the electrosurgical device comprises two electrosurgical instruments 14, 15. The electrosurgical instruments 14, 15 in each case comprise a signal generator 17 for generating a radio-frequency electrical signal that can be transmitted to the tissue of a patient via an active electrode 18 and a neutral electrode 19. In contrast to the embodiment in FIG. 1, the indicator 20 does not communicate the power level, but rather the instantaneous power of the signal generator 17. Since the signal generators 17 are not operated permanently with the maximum power possible within the respective power level, the instantaneous power is regularly lower. In the logic module 22, the power values of the two electrosurgical instruments 14, 15 are added to form a total power value, which can constantly change like the instantaneous power. The logic module 22 constantly compares the total power value with the stored threshold value of 400 W. If the threshold value is exceeded, a signal is passed to the command transmitter 23 and a warning luminar 24 is activated. The command transmitter 23 transmits a control command to one of the electrosurgical instruments 14, 15, such that the relevant electrosurgical instrument reduces the power again. Preferably, for this purpose, one of the electrosurgical instruments 14, 15 is designed as a master and the other as a slave. The control command for reducing the power is transmitted to the slave electrosurgical instrument. This ensures that no surprising reduction of the power can take place at the master electrosurgical instrument.

Alternatively, the electrosurgical device can also be designed such that only the power levels are monitored at a first stage. As long as the power levels in total lie below the threshold value, each signal generator can at any time supply the power that is requested from it within the power level. If the power levels in total exceed the threshold value, a transition is made to a second stage, in which the instantaneous powers of the signal generators are monitored. At the second stage, the users must reckon with the fact that the signal generators temporarily cannot provide the desired power because they are precisely subject to a power restriction.

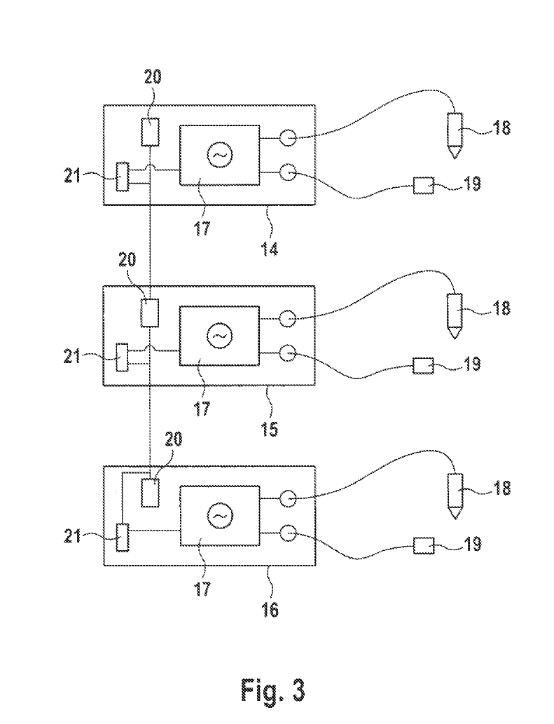

FIG. 3 again shows an embodiment comprising three electrosurgical instruments 14, 15, 16. The indicators 20 of the three electrosurgical instruments 14, 15, 16 are connected to one another, such that the total power value can be requested in each of the electrosurgical instruments 14, 15, 16. Each of the electrosurgical instruments comprises a control module 21, in which the total power value is compared with the threshold value. If the power is intended to be changed in one of the electrosurgical instruments, the control module can ascertain whether the threshold value would be exceeded by this power change. If this is the case, the relevant electrosurgical instrument can be prevented from effecting the power change.

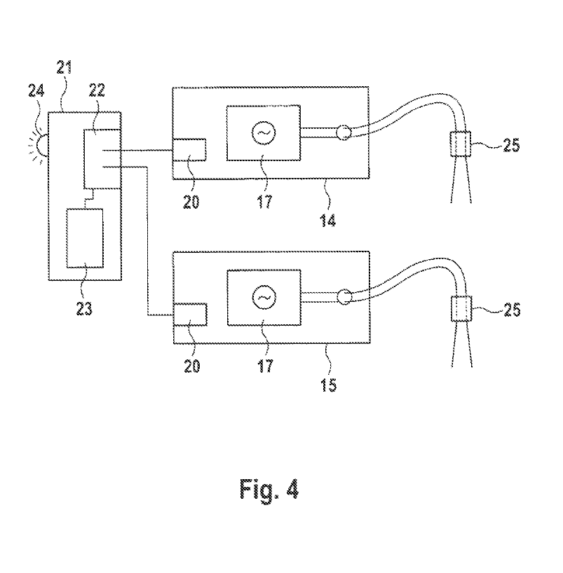

FIG. 4 shows an embodiment which corresponds to FIG. 2 and in which the manual instrument 25 is designed as tweezers that combine both the electrodes therein. When the surgeon moves the manual instrument, said surgeon always guides both electrodes simultaneously. The electrical energy primarily acts on the tissue enclosed between the electrodes. This type of application is designated as bipolar.

1. An electrosurgical device comprising a first signal generator having first electrodes and comprising a second signal generator having second electrodes, wherein the signal generators are designed to generate an electrical signal that can be transmitted to a patient via electrodes, characterized in that a control module is provided, in which a power value of the first signal generator and a power value of the second signal generator are combined to form a total power value and which ascertains whether the total power value exceeds a predefined threshold value.

2. The electrosurgical device as claimed in claim 1, characterized in that more than two signal generators are provided, and in that the control module combined the power values of the more than two signal generators to form a total power value.

3. The electrosurgical device as claimed in claim 1, characterized in that the control module outputs a signal for the operation personnel after the predefined threshold value has been exceeded.

4. The electrosurgical device as claimed in claim 1, characterized in that, after the predefined threshold value has been exceeded, the control module outputs a control command directed to one or more signal generators.

5. The electrosurgical device as claimed in claim 4, characterized in that the signal generator is deactivated by the control command.

6. The electrosurgical device as claimed in claim 4, characterized in that a power limit is set for the signal generator by the control command.

7. The electrosurgical device as claimed in claim 1, characterized in that the control module is designed to combine the power levels of the signal generators to form a total power value.

8. The electrosurgical device as claimed in claim 1, characterized in that the control module is designed to combine instantaneous powers of the signal generators to form a total power value.

9. The electrosurgical device as claimed in claim 8, characterized in that the control module is designed, at a first stage, to combine the power levels of the signal generators to form a total power value and, at a second stage, to combine the instantaneous powers of the signal generators to form a total power value, and in that a change to the second stage is made if the total power value at the first stage has exceeded the threshold value.

10. The electrosurgical device as claimed in claim 1, characterized in that the signal generators are assigned a priority that reveals for which of the signal generators a power limitation is carried out.

11. The electrosurgical device as claimed in claim 1, characterized in that a plurality of control modules are provided, and in that each control module is assigned to a signal generator.

12. A method for operating an electrosurgical device comprising a first signal generator having first electrodes and a second signal generator having second electrodes, comprising the following steps:
a. providing a power value of the first signal generator;
b. providing a power value of the second signal generator;
c. combining the power values to form a total power value; and
d. ascertaining whether the total power value exceeds a predefined threshold value.

13. The electrosurgical device as claimed in claim 2, characterized in that the control module outputs a signal for the operation personnel after the predefined threshold value has been exceeded.

14. The electrosurgical device as claimed in claim 2, characterized in that, after the predefined threshold value has been exceeded, the control module outputs a control command directed to one or more signal generators.

15. The electrosurgical device as claimed in claim 3, characterized in that, after the predefined threshold value has been exceeded, the control module outputs a control command directed to one or more signal generators.

16. The electrosurgical device as claimed in claim 2, characterized in that the control module is designed to combine the power levels of the signal generators to form a total power value.

17. The electrosurgical device as claimed in claim 3, characterized in that the control module is designed to combine the power levels of the signal generators to form a total power value.

18. The electrosurgical device as claimed in claim 4, characterized in that the control module is designed to combine the power levels of the signal generators to form a total power value.

19. The electrosurgical device as claimed in claim 5, characterized in that the control module is designed to combine the power levels of the signal generators to form a total power value.

20. The electrosurgical device as claimed in claim 6, characterized in that the control module is designed to combine the power levels of the signal generators to form a total power value.
